(19) United States
(12) Patent Application Publication
(10) Pub. No.: US 2015/0119809 A1
(43) Pub. Date: Apr. 30, 2015

(54) Medicament Delivery Device

(71) Applicant: Carebay Europe Ltd., Swatar (MT)

(72) Inventor: Stefan Lööf, Skondal (SE)

(73) Assignee: Carebay Europe Ltd., Swatar (MT)

(21) Appl. No.: 14/395,110

(22) PCT Filed: Apr. 9, 2013
(86) PCT No.: PCT/EP2013/057409

§ 371 Date: Oct. 17, 2014

Related U.S. Application Data
(60) Provisional application No. 61/625,820, filed on Apr. 18, 2012.

(30) Foreign Application Priority Data
Apr. 18, 2012 (SE) 1250384-3

(51) Int. Cl. A61M 5/24

(52) U.S. Cl. CPC A61M 5/24; A61M 2005/2437
USPC 604/187

(57) ABSTRACT

A medicament container retaining mechanism for a medicament delivery device includes at least one flexible retaining member arranged to a housing part of the medicament delivery device. The housing part is configured to receive a generally tubular medicament container. The at least one flexible retaining member is configured to interact with an outer surface of a received medicament container, exerting lateral clamping forces on the medicament container to prevent movement of the container in a generally lateral direction in the housing part.

### TECHNICAL AREA

The present invention relates to a medicament delivery device, especially an injection device utilizing a medicament container retaining mechanism to prevent rattling of a medicament container or ampoule containing a specific medicament therein.

### BACKGROUND OF INVENTION

Medicament delivery devices that can be manually loaded with a medicament container or assembled to house a pre-specified cartridge therein are known. Conventional injectors built to accommodate a medicament container therein typically include mechanical structures designed to retain or hold the container within the housing of the injectors. Furthermore, a standard cartridge or ampoule containing a specific medicament therein is known to comprise at least the parts of: (a) an outlet end portion with a septum, (b) a barrel portion for containing the medicament and (c) a neck portion connecting the outlet portion and the barrel portion of the cartridge.

To provide proper injections and to withstand the driving force of a driving mechanism of an auto or semi-auto injector, the container received within the housing of the injector needs to be retained firmly or secured properly such that the force-actuated plunger thereof can be driven, thereby driving a stopper of the medicament container accurately towards the proximal end of the injector such that a correct amount of medicament can be delivered. It is known that by providing a mechanical structure to grip on the neck portion of the cartridge, the cartridge can be secured within the housing of the injector and restricting at least the longitudinal or vertical movement of the medicament container. However, such retaining of the medicament container within the housing of the injector may be sufficient to a relatively short ampoule or a cartridge that is short in length. There is a need to prevent undesirable movement or non-securement of the cartridge received in the injector, in particularly for cartridges of a greater length, such that undesirable movement in the lateral direction, causing rattling of the container in the injector, can be advantageously prevented for proper injections.

### BRIEF DESCRIPTION OF INVENTION

The aim of the present invention is to remedy any drawbacks of medicament delivery devices of the state of the art, and to provide an improved medicament container retaining mechanism.

In the present application, when the term “distal” is used, this refers to the direction pointing away from the dose delivery site. When the term “distal part/end” is used, this refers to the part/end of the delivery device, or the parts/ends of the members thereof, which under use of the medicament delivery device is/are located furthest away from the dose delivery site. Correspondingly, when the term “proximal” is used, this refers to the direction pointing to the dose delivery site. When the term “proximal part/end” is used, this refers to the part/end of the delivery device, or the parts/ends of the members thereof, which under use of the medicament delivery device is/are located closest to the dose delivery site. Further, the term “longitudinal”, with or without “axis”, refers to a direction or an axis through the device or components thereof in the direction of the longest extension of the device or the component. In a similar manner, the term “lateral”, with or without “axis”, refers to a direction or an axis through the device or components thereof in a direction generally perpendicular to the longitudinal direction. Also, if nothing else is stated, in the following description wherein the mechanical structure of the device and the mechanical interconnection of its components is described, the device is in an initial non-activated or non-operated state.

According to a main aspect of the invention, it is characterised by the features of the independent patent claim.

Preferable embodiments of the invention form the subject of the dependent patent claims.

According to the present invention, there is provided a cartridge retaining mechanism for a medicament delivery device. Preferably it comprises at least one flexible retaining member arranged to a housing part of said medicament delivery device. The housing part is configured or designed to receive a generally tubular medicament container therein. The medicament container may be of several types such as syringes, ampules, cartridges, just to mention a few.

According to a preferable aspect of the invention, the at least one flexible retaining member is configured to interact with an outer surface of said medicament container received therein. The at least one flexible retaining member is configured to exert lateral clamping forces to said medicament container to prevent movement in the generally lateral direction of said medicament container in said housing part.

In this manner, the movement of the medicament container is prevented or at least greatly reduced, whereby any rattling or other noises coming from the movement of the medicament container inside the housing is effectively removed or reduced. The device is thereby perceived as being stable and solid by a user, giving a positive appearance of quality.

In order to increase the contact between the retaining member and the medicament container, the flexible retaining member may further comprise a contacting surface of a convex profile shaped to protrude inwardly toward said outer surface of the medicament container. The contacting surface is then generally complimentary to the outer surface of the medicament container.

In a preferable embodiment, the at least one flexible retaining member is arranged at a distal end of said medicament container when placed in said housing part. This position of the retaining member provides an increased stability of the medicament container inside the housing part since the medicament container is often supported well at its proximal end and it is usually only the distal end of the medicament container that is movable in lateral directions.

According to a further preferable solution of the present invention, said at least one flexible retaining member may be integrally formed with said housing part as one unitary part. This solution facilitates the manufacture of the present invention.

In that aspect, said at least one flexible retaining member may be formed by a generally U-shaped cut-out portion in said housing part and arranged such that said at least one retaining member may flex laterally in a direction substantially perpendicular to a longitudinal axis of the housing part to provide said lateral clamping forces on said outer surface of the medicament container. This design also facilitates the manufacturing of the present invention in that the flexible member may be formed as a tongue by the U-shaped cut-out.

These and other aspects of, and advantages with, the present invention will become apparent from the following detailed description of the invention and from the accompanying drawings.

### BRIEF DESCRIPTION OF DRAWINGS

In the following detailed description of the invention, reference will be made to the accompanying drawings, of which

FIG. 1 is a perspective side-view of a medicament delivery device comprising the present invention,

FIG. 2 is a cross-sectional view of a proximal part of the medicament delivery device of FIG. 1, and

FIGS. 3-5 are detailed views of a housing part arranged with a medicament container retaining mechanism according to the present invention.

### DETAILED DESCRIPTION OF THE INVENTION

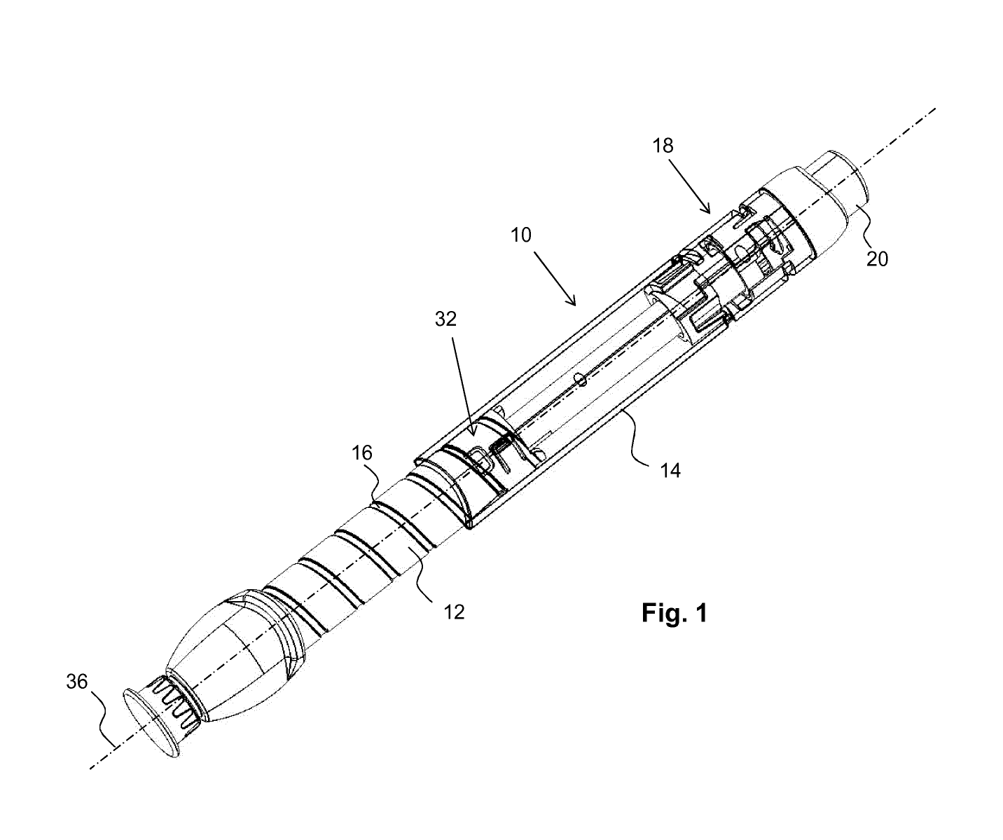

In an exemplary embodiment of a medicament delivery device comprising the present invention, as shown in FIG. 1, the medicament delivery device 10 comprises a proximal housing part 12 having a generally tubular shape, and a distal housing part 14. The proximal housing part 12 is arranged with attachment means arranged to cooperate with corresponding attachment means on the distal housing part 14. As an example, in the embodiment shown the attachment means on the proximal housing part 12 comprises a spirally extending groove 16 arranged on its outer surface. The groove cooperates with a mating structure (not shown), capable of running in the groove, on an inner surface of the distal housing part 14, such that the two housing parts may be screwed together. It is however to be understood that a number of other types of attachment means are available to the person skilled in the art, such as bayonet connections, snap-in members etc.

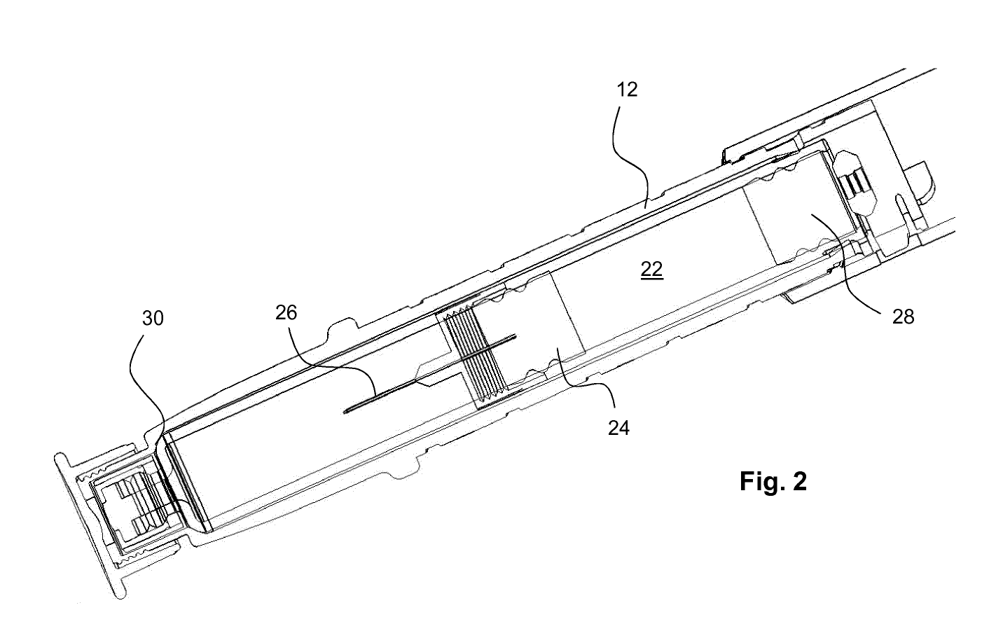

The distal housing part 14 further comprises a drive mechanism 18, which drive mechanism may have an activation member 20 at a distal end of the distal housing part 14. Further, the proximal housing part 12 is designed to accommodate a medicament container 22 containing a specific medicament therein, FIG. 2. As a non-limiting example, the medicament container may be a so-called dual chamber container arranged with two compartments, one with the medicament in powder form and the other with a diluent. The compartments are initially closed by a first generally resilient stopper 24, which also may comprise a delivery member, such as an injection needle 26. At a distal end of the medicament container a second stopper 28 is arranged. It is however to be understood that the type of medicament container is not vital for the present invention. The medicament container may as well comprise a single chamber medicament container where the medicament is prepared and ready for delivery.

A proximal end surface 30 of the proximal housing part is preferably arranged to be in contact with a neck portion of the medicament container 22, providing a stop or reference surface for the medicament container in the proximal direction of the device.

According to the present invention, it comprises a medicament container retaining mechanism 32, FIG. 1. The medicament container retaining mechanism is arranged on the proximal housing part 12 to facilitate securement of a medicament container 22 entered into the proximal housing part from a distal direction, and to prevent or avoid movement in a generally lateral direction, preventing rattling of the medicament container 22 in the proximal housing part 12.

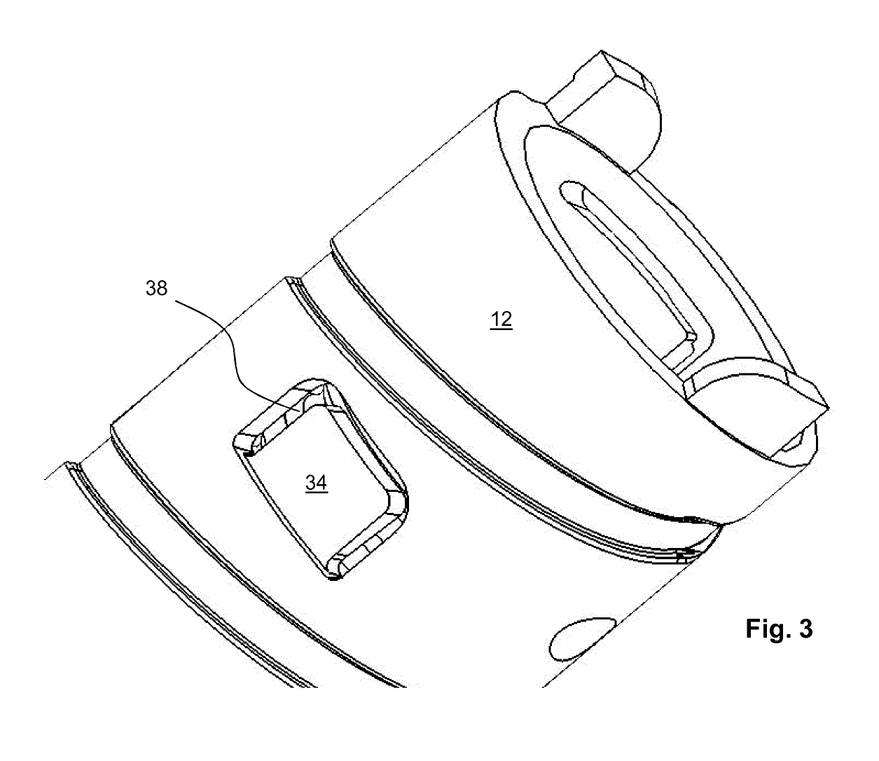

As shown in FIG. 1, the medicament container retaining mechanism 32 is provided on at least a distal portion of the proximal housing part 12. In a preferred embodiment shown, the proximal housing part 12 comprises at least one flexible retaining member 34, FIG. 3, preferably two positioned on opposite sides as seen along a longitudinal axis 36, configured to interact or detachably engage with an outer surface of the generally tubular medicament container 22.

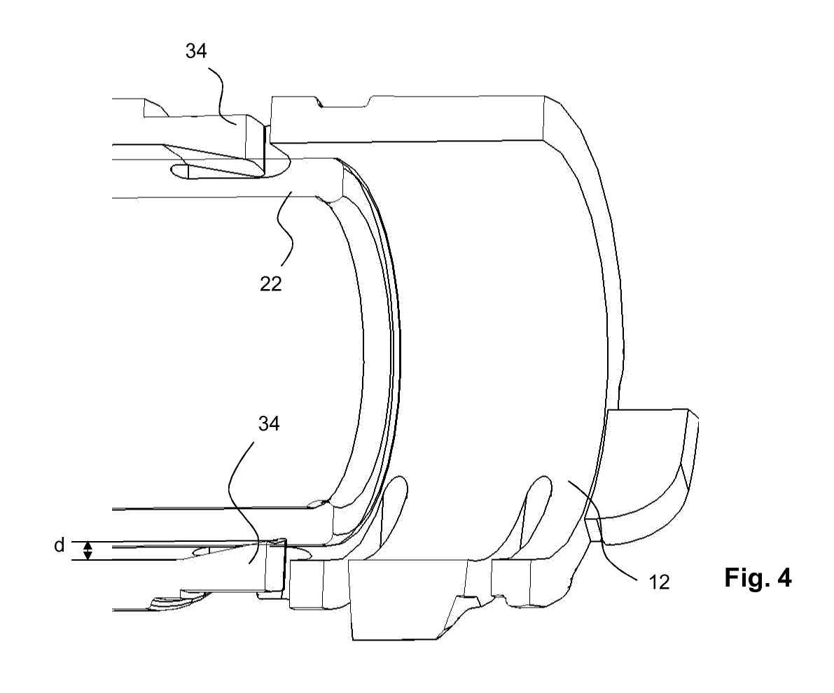

The retaining members 34 are in the embodiment shown formed integral with the material of the proximal housing part 12 where each retaining member is formed by a generally U-shaped cut-out 38, FIG. 3, whereby a tongue is formed, extending in the generally distal direction of the proximal housing part 12. The tongues are designed to protrude radially inwards a distance d more than the inner surface of the proximal housing part, FIG. 4. In the embodiment shown this radial protrusion is obtained in that the tongue is given a gradually larger thickness as seen in FIG. 4. It is however to be understood that other designs and features are possible, such as inwardly extending protrusions at the free end of the tongue, or that the tongues have an inclination inwards or combinations of features.

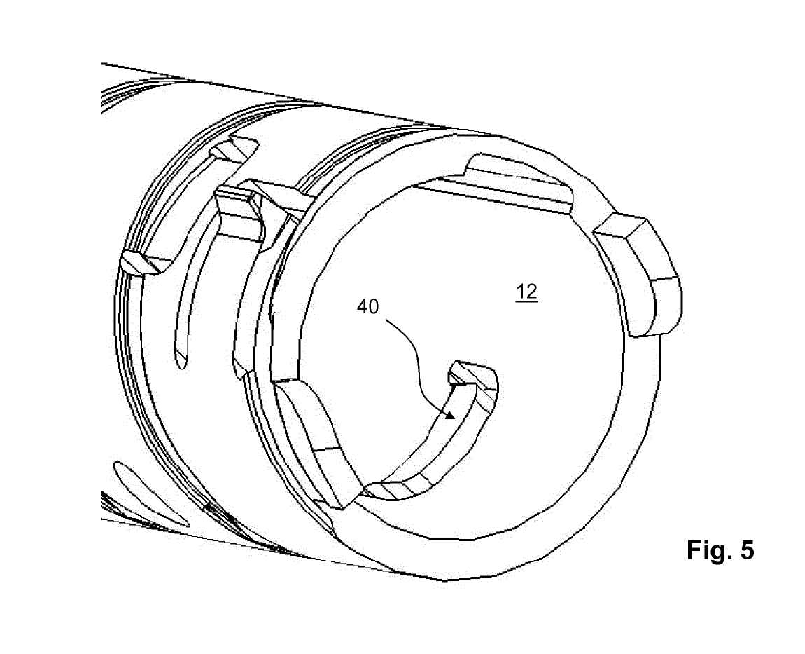

The free ends of the tongues are arranged with a contacting surface 40, FIG. 5, having a convex profile shaped to protrude inwardly toward the outer surface of the medicament container 22 placed inside the proximal housing part. It is however to be understood that other shapes and profiles of the contacting surface 40 of the retaining members 34 can be utilized with maintained lateral clamping force on the medicament container 22 placed inside the proximal housing part.

Due to the mechanical structural configurations of the retaining members 34, the retaining members 34 are able to flex laterally in a direction substantially perpendicular to the longitudinal axis 36 of the proximal housing part 12 to provide lateral clamping forces on the outer surface medicament container 22 received therein, preventing lateral movement and thereby rattling of said medicament container 22 in the proximal housing part 12. In this context, it is to be understood that the arrangement and location of the cartridge retaining mechanism 32 may be performed on other portions along the lateral sides of the proximal housing part.

The proximal housing part 12 including the retaining member 34 may be made of flexible materials including such as plastics, metal alloys and combinations thereof, whereby the retaining member 34 of the cartridge retaining mechanism 32 exhibits a resilient property by the properties of the material in order to be able to flex in a direction substantially perpendicular to the longitudinal axis 36 of the proximal housing part 12. It is to be understood that the number of retaining members 34 of the cartridge retaining mechanism 32 is not limited to any number, and there may be provided with more than one pair of retaining members 34 or an odd number of said members 34 on the proximal housing part 12 of the medicament delivery device 10.

It is to be understood that the embodiment described above and shown in the drawings is to be regarded only as a non-limiting example of the invention and that it may be modified in many ways within the scope of the patent claims.

1.-4. (canceled)

5. A medicament container retaining mechanism for a medicament delivery device, comprising:
at least one flexible retaining member configured to attach to a housing part of the medicament delivery device, the housing part being configured to receive a generally tubular medicament container therein;
wherein the at least one flexible retaining member is configured to interact with an outer surface of a received medicament container, thereby exerting a clamping force on the received medicament container to prevent movement of the received medicament container in the housing part in a substantially lateral direction that is substantially perpendicular to a longitudinal axis of the housing part, and the at least one flexible retaining member is a substantially U-shaped cutout integral with the housing part such that the at least one retaining member flexes in the lateral direction, thereby exerting the clamping force on an outer surface of the medicament container.

6. The medicament container retaining mechanism of claim 5, wherein the at least one flexible retaining member is arranged at a distal end of the medicament container when received in the housing part.

7. The medicament container retaining mechanism of claim 5, wherein the at least one flexible retaining member further comprises a contacting surface having a convex profile shaped to protrude inward toward the outer surface of the medicament container.

8. The medicament container retaining mechanism of claim 7, wherein the at least one flexible retaining member is arranged at a distal end of the medicament container when received in the housing part.

9. The medicament container retaining mechanism of claim 5, wherein the at least one cut-out portion is provided on a top portion of the at least one flexible retaining member.
